(19) United States
(12) Patent Application Publication
(10) Pub. No.: US 2023/0051424 A1
(43) Pub. Date: Feb. 16, 2023

(54) PROCESS FOR THE PREPARATION OF A DENTAL RESTORATION

(71) Applicant: Ivoclar Vivadent AG, Schaan (LI)

(72) Inventors: Corina Serban, Lindau (DE); Frank Rothbrust, Röns (AT); Christian Ritzberger, Grabs (CH); Sebastian Krolikowski, Lachen (CH)

(21) Appl. No.: 17/819,812

(22) Filed: Aug. 15, 2022

(30) Foreign Application Priority Data
Aug. 16, 2021 (EP) 21191535.0

(51) Int. Cl. C04B 35/64; A61C 13/00; C04B 35/48

(52) U.S. Cl. CPC C04B 35/64; A61C 13/0003; C04B 35/48; A61C 2201/00; C04B 2235/3246; C04B 2235/3229; C04B 2235/3206; C04B 2235/3208; C04B 2235/6565; C04B 2235/6567; C04B 2235/6581; C04B 2235/9661

(57) ABSTRACT

The invention relates to a process for the preparation of a dental restoration, in which an oxide ceramic material is
(a) subjected to at least one heat treatment, and
(b) cooled,
wherein the cooling comprises
(b1) a first cooling step with the cooling rate T1 and
(b2) a second cooling step with the cooling rate T2
and wherein the absolute value of the cooling rate T2 is less than the absolute value of the cooling rate T1.

### CROSS-REFERENCE TO RELATED APPLICATIONS

This application claims priority to European patent application No. 21191535.0 filed on Aug. 16, 2021, which disclosure is incorporated herein by reference in its entirety.

### TECHNICAL FIELD

The present invention relates to a process which, starting from an oxide ceramic material, enables the production of a dental restoration with excellent properties in a short time. The invention also relates to the use of an oxide ceramic material for the preparation of a dental restoration by means of the process according to the invention.

### BACKGROUND

Ceramic materials such as oxide ceramics are frequently used to fabricate fully anatomical dental restorations. They offer a high degree of clinical safety, are usually metal-free, can also be used for minimally invasive preparations and are very attractive in terms of price compared to other metal-free restorations. However, the numerous working steps that are usually required to fabricate such restorations are a disadvantage.

The restorations are usually milled or ground from presintered blanks, shaded if necessary, densely sintered by thermal treatment and finally further shaded, glazed and/or polished if necessary.

Conventional sintering processes for dental ceramics involve slow heating to a maximum temperature at which the employed oxide ceramic material is densely sintered. Due to the low heating rate, such a sintering process typically takes considerably more than 4 hours and thus contributes significantly to the, especially in chairside treatments, unsatisfactorily long duration of the production cycle of dental ceramics.

Approaches to accelerate the sintering process by increasing the heating rate are generally known.

For example, EP 2 098 188 A1 and corresponding U.S. Pat. No. 9,033,703, which US patent is incorporated by reference in its entirety, describe a dental furnace and a method for sintering dental materials, in which the furnace is heated to a presintering temperature of at least 1000° C. in a first heating period at a rapid heating rate of more than 50 K/min.

EP 2 101 133 A1 describes a sintering furnace and a process for sintering dental articles, in which the dental articles are moved along a sintering section and exposed to different temperatures. High heating rates of 300 K/min or more can be used in a first section.

WO 2012/057829 A2 and corresponding U.S. Pat. No. 8,845,951, which US patent is incorporated by reference in its entirety, describe a process for rapid sintering of ceramics using electromagnetic induction or a plasma.

WO 2015/091744 A1 and corresponding U.S. Ser. No. 10/939,980, which US patent is incorporated by reference in its entirety, describe a process for planning the sintering of a dental prosthesis part, in which a temperature profile for the heat treatment of the dental prosthesis part is automatically determined by means of a computer as a function of certain geometry and material parameters of the dental prosthesis part to be produced. A heating rate between 100 K/min and 400 K/min is used for the sintering of certain dental prosthesis parts.

WO 2015/121364 A1 and corresponding U.S. Ser. No. 11/306,969, which US patent is incorporated by reference in its entirety, describe a sintering furnace for dental components with a heating device that enables a heating rate of at least 200 K/min in the useful range.

The sintering of dental materials under inert gas or in a vacuum is also known.

WO 2011/020688 A1 describes a device for oxygen-free sintering of metal or ceramics in dental technology under inert gas.

EP 2 703 760 A1 describes a dental furnace for sintering a dental prosthesis, the heating chamber of which can be closed either by means of a conventional closing device such as a door or by means of an attachment with a vacuum container and can thus be used either for normal sintering or for vacuum sintering.

WO 2017/189414 A1 and corresponding U.S. Ser. No. 11/253,436, which US patent is incorporated by reference in its entirety, describe a method of manufacturing a dental restoration comprising an additional heat treatment to induce a color change.

EP 3 659 548 A1 and corresponding US 2020170765, which US published application is incorporated by reference in its entirety, describe a process of manufacturing a dental restoration in which an oxide ceramic material is subjected to at least two heat treatments, the first heat treatment being carried out at a lower pressure than the second heat treatment.

However, it has been shown that the known approaches to speeding up the sintering process lead to ceramic materials whose properties do not meet the high requirements in the dental field, especially in terms of optical properties.

### SUMMARY

The invention is therefore based on the problem of providing a process for the preparation of a dental restoration by which dental restorations with excellent mechanical and, in particular, optical properties can be produced in a short time by sintering oxide ceramic material.

### DETAILED DESCRIPTION

The process for the preparation of a dental restoration according to the invention is characterized in that an oxide ceramic material is

(a) subjected to at least one heat treatment, and
(b) cooled,
wherein the cooling comprises
(b1) a first cooling step with the cooling rate T1 and
(b2) a second cooling step with the cooling rate T2
and wherein the absolute value of the cooling rate T2 is less than the absolute value of the cooling rate T1.

It was surprisingly found that the process according to the invention allows very fast sintering of oxide ceramics to dental restorations, which have good mechanical properties and in particular a high density and at the same time can meet the high aesthetic requirements of dental restorations and imitate the optical properties of natural dental material in an excellent manner.

The oxide ceramic material used in step (a) is usually a non-densely sintered and in particular a presintered oxide ceramic material. Typically, the oxide ceramic material used has a relative density in the range from 30 to 90%, in particular in the range from 40 to 80% and preferably in the range from 50 to 70%, in each case based on the true density of the oxide ceramic material.

The relative density is the ratio of the apparent density of the oxide ceramic material to the true density of the oxide ceramic material.

The apparent density of the oxide ceramic material can be determined by weighing it and determining its volume geometrically. The density is then calculated according to the known formula

Density=Mass/Volume.

The determination of the true density of the oxide ceramic material is carried out by grinding the oxide ceramic material to a powder with an average particle size of 10 to 30 μm, in particular 20 μm, based on the number of particles, and determining the density of the powder by means of a pycnometer. The particle size can be determined, for example, with the CILAS® Particle Size Analyzer 1064 from Quantachrome GmbH & Co. KG using laser diffraction according to ISO 13320 (2009).

In a preferred embodiment of the process, the cooling comprises

(b1) a first cooling step with the cooling rate T1,
(b2) a second cooling step with the cooling rate T2 and
(b3) a third cooling step with the cooling rate T3,
wherein the absolute value of the cooling rate T2 is less than the absolute values of the cooling rates T1 and T3.

Furthermore, it is preferred according to the invention that step (b2), in which the absolute value of the cooling rate T2 is smaller than the absolute values of the cooling rate T1 and optionally of the cooling rate T3, takes place in a temperature range from 1000 to 1500° C., preferably 1100 to 1400° C. and particularly preferably 1200 to 1300° C.

It is also preferred that in step (b2) the absolute value of cooling rate T2 is less than 60 K/min, preferably less than 50 K/min, more preferably less than 40 K/min, further preferably less than 25 K/min, even more preferably less than 10 K/min and most preferably less than 5 K/min. In a particularly preferred embodiment of the process, step (b2) is carried out at a substantially constant temperature such that the cooling rate T2 is about 0 K/min.

Step (b2) can be carried out in particular for a duration of 1 to 20 min, preferably 1 to 10 min, more preferably 2 to 8 min, particularly preferably 3 to 7 min and most preferably 4 to 6 min.

According to the invention, the absolute value of the cooling rate T2 is smaller than the absolute values of the cooling rate T1 and optionally of the cooling rate T3. In a preferred embodiment, the absolute value of the cooling rate T1 is at least 40 K/min, preferably at least 50 K/min and particularly preferably at least 60 K/min and is in particular in the range of 40 to 200 K/min, preferably 50 to 100 K/min and particularly preferably 60 to 80 K/min. In another preferred embodiment, the absolute value of the cooling rate T3 is at least 40 K/min, preferably at least 50 K/min and particularly preferably at least 60 K/min and is in particular in the range of 40 to 200 K/min, preferably 50 to 100 K/min and particularly preferably 60 to 80 K/min. In a particularly preferred embodiment, the absolute values of the cooling rates T1 and T3 are each at least 40 K/min, preferably at least 50 K/min and particularly preferably at least 60 K/min and are in particular in the range from 40 to 200 K/min, preferably 50 to 100 K/min and particularly preferably 60 to 80 K/min.

In step (a), the oxide ceramic material is preferably heated to a temperature which is in the range of 1100 to 1700° C., preferably in the range of 1300 to 1600° C., more preferably in the range of 1400 to 1550° C., and particularly preferably in the range of 1450 to 1500° C., and most preferably is about 1480° C. It is further preferred that the oxide ceramic material after carrying out step (a) has a relative density in the range of 90 to 97%, preferably in the range of 93 to 96% and particularly preferably a relative density of about 95%, in each case based on the true density of the oxide ceramic material.

Preferably, the oxide ceramic material is heated in step (a) at a heating rate in the range of 5 to 500 K/min, preferably 50 to 250 K/min and particularly preferably 100 to 200 K/min. In a preferred embodiment, the oxide ceramic material is first heated at a heating rate of 50 to 500 K/min, preferably 75 to 250 K/min and particularly preferably 100 to 200 K/min to a temperature which is 100 to 800 K, preferably 300 to 700 K and particularly preferably 500 to 600 K below the maximum temperature reached in step (a), and then further heated at a heating rate of 5 to 200 K/min, preferably 10 to 100 K/min and particularly preferably 25 to 50 K/min.

In step (b), the oxide ceramic material is preferably cooled to a temperature which is in the range of 20 to 1300° C., preferably in the range of 100 to 1250° C., and particularly preferably in the range of 1000 to 1200° C. After reaching such a temperature, the oxide ceramic material can be removed from the heating chamber.

According to a further embodiment, a process is particularly preferred in which the oxide ceramic material in step (a) is

(a1) subjected to a first heat treatment, and
(a2) subjected to a second heat treatment,
wherein the heat treatment in step (a1) is carried out at a lower pressure than the heat treatment in step (a2).

Preferably, the heat treatment in step (a1) is carried out at a pressure of less than 200 mbar, preferably less than 100 mbar and particularly preferably less than 50 mbar, and in particular at a pressure in the range from 0.1 to 200 mbar, preferably in the range from 1 to 150 mbar and particularly preferably in the range from 50 to 100 mbar.

This pressure can be set at ambient temperature before starting to heat the oxide ceramic material. Alternatively, the oxide ceramic material may first be heated to a temperature above ambient temperature before the pressure defined for step (a) is adjusted. This temperature is preferably in the range of 20 to 500° C., and in particular in the range of 25 to 100° C.

In step (a2), the oxide ceramic material is preferably further heated and held and sintered at a, preferably constant, temperature in the range of 1100 to 1700° C., in particular in the range of 1300 to 1600° C., preferably in the range of 1400 to 1550° C., more preferably in the range of 1450 to 1500° C. and most preferably at a temperature of about 1480° C. The further heating is preferably carried out at a heating rate of 5 to 200 K/min, in particular 10 to 100 K/min and preferably 25 to 50 K/min. The holding is preferably carried out for 1 to 60 minutes, in particular 5 to 30 minutes, preferably 10 to 25 minutes and particularly preferably 15 to 20 minutes. By holding at the corresponding temperature, the oxide ceramic material is typically densely sintered. Thereafter, the oxide ceramic material preferably has a relative density of at least 97%, in particular at least 98%, preferably at least 99% and most preferably at least 99.5%, in each case based on the true density of the oxide ceramic material.

The heat treatment in step (a2) is preferably carried out at a pressure greater than 500 mbar and in particular at ambient pressure.

Preferably, the heat treatment in step (a2) is carried out in an oxygen-containing atmosphere. In particular, air, oxygen-enriched air as well as oxygen can be considered as an oxygen-containing atmosphere. To set such an atmosphere, the heating chamber used for the heat treatment can be filled with air and/or oxygen. In a preferred embodiment, an oxygen-containing atmosphere, preferably air, oxygen-enriched air or oxygen, flows discontinuously or preferably continuously through the heating chamber used for the heat treatment during step (a2), in particular at a flow rate of 0.1 to 50 l/min, preferably 1 to 10 l/min and particularly preferably 2 to 5 l/min.

It is also preferred that the oxide ceramic material is heated in step (a1) to a temperature that is 0 to 500 K, in particular 10 to 250 K, preferably 50 to 150 K, and more preferably 75 to 100 K below the temperature or temperature range at which the oxide ceramic material is maintained in step (a2).

The oxide ceramic material obtained by the process according to the invention preferably has a number-average grain size in the range from 1 nm to 1000 nm, in particular from 10 nm to 800 nm and preferably from 100 nm to 600 nm. The number-average grain size can be determined in particular by the line intersection method according to DIN EN 623-3 or ASTM E 112, wherein the value determined is used for conversion to the real number-average grain size in the three-dimensional microstructure according to M. I. Mendelson, J. Am. Ceram. Soc. 1969, 52(8), 443-446 by multiplying it by a proportionality constant of 1.56.

The process according to the invention is suitable for various types of oxide ceramic materials. Oxide ceramic materials are generally highly crystalline ceramic materials based on oxide compounds and are having at most a very small proportion of glass phase. Typical oxide ceramic materials are based on ZrO₂, Al₂O₃, TiO₂, MgO, combinations, solid solutions or composites thereof, in particular ZrO₂/Al₂O₃(ZTA), Al₂O₃/ZrO₂ (ATZ) or ZrO₂/spinel, with spinel preferably being Sr-spinel, Mg-spinel, La-spinel and/or Ce-spinel. Oxide ceramic materials based on ZrO₂ and/or Al₂O₃ are preferred according to the invention.

Oxide ceramic materials based on zirconium oxide and in particular based on polycrystalline tetragonal zirconium oxide (TZP) are particularly preferred. Even more preferred are oxide ceramic materials based on zirconium oxide in which the zirconium oxide is stabilized with Y₂O₃, La₂O₃, CeO₂, MgO and/or CaO and is preferably stabilized with 2 to 12 mol %, in particular 3 to 6 mol %, of these oxides, based on the zirconium oxide content.

It is further preferred that the oxide ceramic material is coloured. According to the invention, this refers to an oxide ceramic material to which one or more colouring elements have been added. Examples of suitable colouring elements are Fe, Mn, Cr, Pr, Tb, Er, Yb, Ce, Co, Ni, Nd, Cu and Bi. Preferably, the oxide ceramic material in particular comprises Fe. Particularly preferably, the oxide ceramic material comprises at least two layers which differ in particular in their color.

Within the meaning of the present application, the terms “colour” and “coloured” refer to the colour, brightness and/or translucency of a material.

“Translucency” is the light transmission of a material, body or layer, i.e. the ratio of transmitted to incident light intensity.

Colors can also be characterized by the color coordinates L*, a* and b* in the L*a*b* color space or by a color code commonly used in the dental industry.

In the L*a*b* color space, the L* value describes the brightness of a color with values from 0 (black) to 100 (white), the a* value describes the green or red component of a color, where negative values represent green and positive values represent red, and the b* value describes the blue or yellow component of a color, where negative values represent blue and positive values represent yellow. Color differences can be expressed in the L*a*b* color space by the ΔE* value, which is calculated as follows:

ΔE*=√((ΔL*)²(Δa*)²(Δb*)²).

Examples of color codes commonly used in the dental industry are Vitapan Classical® and Vita 3D Master®, both from VITA Zahnfabrik H. Rauter GmbH & Co. KG, and Chromascop® from Ivoclar Vivadent AG. The translucency can be characterized by the contrast value CR, where 0% means completely transparent and 100% completely opaque.

Usually, the color coordinates L*, a* and b* are determined according to DIN 5033 and DIN 6174 and the translucency according to BS 5612. The corresponding measurements can be carried out in particular by means of a spectrophotometer of the type CM-3700d (Konica-Minolta). For this purpose, specimens are used for the measurements which have been wet ground on both sides with diamond particles (particle size 15-20 μm) to obtain a final specimen thickness of 2.00±0.025 mm.

Preferably, the color or colors of the dental restoration obtained according to the invention are in the range of the colors of natural teeth. Particularly preferably, the dental restoration obtained according to the invention has an L* value in the range from 50 to 100, in particular in the range from 80 to 97, an a* value in the range from −10 to 10, in particular in the range from −1 to 5, a b* value in the range from 0 to 50, in particular in the range from 1 to 20, and/or a CR value in the range from 50 to 100%, in particular in the range from 75 to 99%.

The process according to the invention is particularly suitable for the preparation of dental restorations. Particularly preferred dental restorations are bridges, inlays, onlays, crowns, veneers, facets and abutments. The process according to the invention is particularly suitable for the preparation of dental restorations, in particular bridges, which comprise two or more members.

The invention also relates to the use of an oxide ceramic material for the preparation of a dental restoration, in which the oxide ceramic material is

(a) subjected to at least one heat treatment, and
(b) cooled,
wherein the cooling comprises
(b1) a first cooling step with the cooling rate T1 and
(b2) a second cooling step with the cooling rate T2
and wherein the absolute value of the cooling rate T2 is less than the absolute value of the cooling rate T1.

Preferred embodiments of the use are as described above for the process according to the invention.

The invention will be explained in more detail below with reference to examples.

### EXAMPLES

### Examples 1A to 1F

Test specimens with a diameter of 24 mm and a height of 2.9 mm were fabricated from a commercial zirconia-based oxide ceramic material containing 9.23 wt.-% Y₂O₃, 0.045 wt.-% Al₂O₃, and 0.25 wt.-% Fe₂O₃ (Zpex smile yellow from the company Tosoh) by uniaxial pressing at a pressure of 150 MPa and heat treatment at 1000° C. for 2 hours.

The test specimens were sintered in a sintering furnace with MoSi₂ heating element. For this purpose, the test specimen was placed in the heating chamber of the sintering furnace at room temperature, the heating chamber was closed and a partial vacuum with a final pressure of about 50 to 100 mbar was created in the heating chamber. The test specimen was heated at a heating rate of about 130 K/min to a temperature of about 900° C., then at a heating rate of about 50 K/min to a temperature of about 1220° C., and further at a heating rate of about 10 K/min to a temperature of about 1400° C. Upon reaching this temperature, the heating chamber was flooded with fresh air and then continuously flowed with fresh air at a flow rate of about 2.2 l/min while the test specimen was further heated to a temperature of 1480° C. at a heating rate of about 10 K/min and held at this temperature for about 17 minutes. Thereafter, the test specimen was cooled to a temperature T at a cooling rate of about 70 K/min according to Table 1, held at this temperature for a period of t minutes, and then further cooled to a temperature of about 1200° C. at a cooling rate of about 70 K/min. The heating chamber was then opened. The total duration of the sintering process was between 64 and 66 min.

### Example 1G (Comparison)

Example 1A was repeated, but the specimen was cooled from 1480° C. to 1200° C. at a continuous cooling rate of about 70 K/min without interruption. The total duration of the sintering process was about 60 min.

### Example 1H (Comparison)

Example 1A was repeated, but using a slow sintering process. For this purpose, the test specimen was heated at a heating rate of about 10 K/min to a temperature of about 900° C., held at this temperature for 30 min, further heated at a heating rate of about 3 K/min to a temperature of about 1500° C. and held at this temperature for about 120 min. Thereafter, the test specimen was cooled to 900° C. at a cooling rate of about 10 K/min and further cooled to 300° C. at a cooling rate of about 8 K/min. The heating chamber was then opened. The total duration of the sintering process was about 575 min.

The CR values and color coordinates of the oxide ceramic materials obtained in Examples 1A-H are shown in Table 1. It can be seen that Examples 1A to 1F according to the invention show significantly higher a* values and b* values compared to Example 1G, which was also obtained using a rapid sintering process, and that the a* values are consistently in the positive range. As a result, these examples are more suitable for imitating the color properties of natural dental material. At the same time, the sintering process used for examples 1A to 1F according to the invention requires only about one tenth of the duration of the slow sintering process according to example 1H.

TABLE 1

| | Sintering | Vacuum up | Cooling | | | | | |
|---|---|---|---|---|---|---|---|---|
| Example | time [min] | to [° C.] | T [° C.] | t [min] | CR [%] | L* | a* | b* |
| 1A | 64 | 1400 | 1200 | 4 | 98.84 | 71.46 | 0.74 | 18.44 |
| 1B | 66 | 1400 | 1200 | 6 | 90.80 | 75.48 | 0.70 | 19.31 |
| 1C | 64 | 1400 | 1250 | | 98.99 | 71.85 | 0.82 | 18.68 |
| 1D | 66 | 1400 | 1250 | 6 | 99.29 | 75.08 | 1.14 | 19.49 |
| 1E | 64 | 1400 | 1300 | 4 | 98.21 | 71.03 | 1.18 | 19.58 |
| 1F | 66 | 1400 | 1300 | 6 | 99.79 | 74.80 | 0.63 | 19.14 |
| 1G* | 60 | 1400 | — | — | 99.32 | 74 .05 | −0.60 | 16.31 |
| 1H* | 575 | — | — | — | 98.60 | 77.98 | 3.03 | 20.74 |

*(comparison)

1. A process for preparing a dental restoration, in which an oxide ceramic material
(a) is subjected to at least one heat treatment, and
(b) is cooled,
wherein the cooling comprises
(b1) a first cooling step with the cooling rate T1 and
(b2) a second cooling step with the cooling rate T2
and wherein an absolute value of the cooling rate T2 is less than an absolute value of the cooling rate T1.

2. The process according to claim 1, in which the cooling comprises
(b1) a first cooling step with the cooling rate T1,
(b2) a second cooling step with the cooling rate T2 and
(b3) a third cooling step with the cooling rate T3,
wherein the absolute value of the cooling rate T2 is less than the absolute values of the cooling rates T1 and T3.

3. The process according to claim 1, in which step (b2) is carried out in a temperature range of 1000 to 1500° C.

4. The process according to claim 1, in which in step (b2) the absolute value of the cooling rate T2 is less than 60 K/min.

5. The process according to claim 1, in which step (b2) is carried out at a substantially constant temperature.

6. The process according to claim 1, in which (b2) is carried out for a duration of 1 to 20 min.

7. The process according to claim 2, in which the absolute value of the cooling rate T1 and/or the absolute value of the cooling rate T3 is at least 40 K/min.

8. The process according to claim 1, in which the oxide ceramic material is heated in step (a) to a temperature which is in the range of 1100 to 1700° C.

9. The process according to claim 1, in which the oxide ceramic material is heated in step (a) at a heating rate in the range of 5 to 500 K/min.

10. The process according to claim 1, in which the oxide ceramic material is cooled in step (b) to a temperature which is in the range of 20 to 1300° C.

11. The process according to claim 1, in which the oxide ceramic material in step (a) is
(a1) subjected to a first heat treatment, and
(a2) subjected to a second heat treatment,
wherein the heat treatment in step (a1) is carried out at a lower pressure than the heat treatment in step (a2).

12. The process according to claim 11, in which the heat treatment in step (a1) is carried out at a pressure of less than 200 mbar in the range from 0.1 to 200 mbar.

13. The process according to claim 11, in which the oxide ceramic material is in step (a2) further heated and held and sintered at a, constant, temperature in the range of 1100 to 1700° C., wherein the holding is effected for 1 to 60 minutes.

14. The process according to claim 11, in which the heat treatment in step (a2) is carried out at a pressure greater than 500 mbar and at ambient pressure and/or in an oxygen-containing atmosphere.

15. The process according to claim 14, in which during step (a2) an oxygen-containing atmosphere is flowed discontinuously or continuously through the heating chamber, at a flow rate of 0.1 to 50 l/min.

16. The process according to claim 11, in which the oxide ceramic material is heated in step (a1) to a temperature which is 0 to 500 K below the temperature or temperature range at which the oxide ceramic material is held in step (a2).

17. The process according to claim 1, in which the oxide ceramic material is based on zirconia.

18. The process according to claim 17, in which zirconia comprises zirconium oxide stabilized with 2 to 12 mol % of Y₂O₃, CeO₂, MgO and/or CaO, based on the amount of zirconium oxide.

19. The process according to claim 1, in which the oxide ceramic material is coloured and comprises at least two layers which differ in colour.

20. The process according to claim 1, in which the oxide ceramic material comprises at least one coloring element selected from the group consisting of Fe, Mn, Cr, Pr, Tb, Er, Yb, Ce, Co, Ni, Nd, Cu, and Bi.

21. The process according to claim 1, in which the dental restoration is a bridge, an inlay, an onlay, a crown, a veneer, a facet or an abutment.

22. A process of using an oxide ceramic material for the preparation of a dental restoration, in which the oxide ceramic material is
(a) subjected to at least one heat treatment, and
(b) cooled,
wherein the cooling comprises
(b1) a first cooling step with the cooling rate T1 and
(b2) a second cooling step with the cooling rate T2
and wherein the absolute value of the cooling rate T2 is less than the absolute value of the cooling rate T1.
